(19) United States
(12) Patent Application Publication
(10) Pub. No.: US 2024/0013887 A1
(43) Pub. Date: Jan. 11, 2024

(54) SCHEDULE MANAGEMENT SYSTEM, ITS CONTROL METHOD, AND CONTROL PROGRAM

(71) Applicant: TOYOTA JIDOSHA KABUSHIKI KAISHA, Toyota-shi (JP)

(72) Inventors: Issei NAKASHIMA, Katsushika-ku (JP); Toru MIYAGAWA, Seto-shi (JP); Hideo HASEGAWA, Nagoya-shi (JP); Yusuke KINOSHITA, Tokyo-to (JP); Takuhiro OMI, Anjo-shi (JP); Yuta MORI, Urayasu-shi (JP); Hajime KATSUDA, Nagareyama-shi (JP)

(73) Assignee: TOYOTA JIDOSHA KABUSHIKI KAISHA, Toyota-shi (JP)

(21) Appl. No.: 18/214,943

(22) Filed: Jun. 27, 2023

(30) Foreign Application Priority Data
Jul. 5, 2022 (JP) 2022-108252

(51) Int. Cl. G16H 20/30; G06Q 10/0631

(52) U.S. Cl. CPC G16H 20/30; G06Q 10/063116

(57) ABSTRACT

A schedule management system includes an acquisition unit configured to acquire information about a plurality of task items and information about a target exercise amount of a first user for each predetermined period; a task dividing unit configured to extract at least one task item from among the plurality of task items by referring to a database, and assign the extracted at least one task item to the first user as a task item for the predetermined period, the at least one task item representing a task with which the first user achieves an exercise amount corresponding to his/her target exercise amount for each predetermined period, and in the database, information about, for each of the plurality of task items, an exercise amount per unit time corresponding to that task item being registered; and an output unit that outputs information about the task item assigned to the first user.

### CROSS REFERENCE TO RELATED APPLICATIONS

This application is based upon and claims the benefit of priority from Japanese patent application No. 2022-108252, filed on Jul. 5, 2022, the disclosure of which is incorporated herein in its entirety by reference.

### BACKGROUND

The present disclosure relates to a schedule management system, its control method, and a control program.

In recent years, it has been desired, for example, for workers in workplaces, to achieve both the performance of tasks assigned to them and exercises for maintaining their good health. For example, Japanese Unexamined Patent Application Publication No. 2018-84911 discloses a related technology.

Japanese Unexamined Patent Application Publication No. 2018-84911 discloses a worker information detection system that determines that a worker is in a warning state when a cumulative load determined from load information of the worker exceeds a threshold.

### SUMMARY

However, the system disclosed in Japanese Unexamined Patent Application Publication No. 2018-84911 merely issues a warning when there is a possibility that a worker (a user) is forced to work too much, and does not assign, to the worker, a task item with which the worker can achieve an appropriate amount of exercise (hereinafter also referred to as an “exercise amount”). Therefore, there is a problem in the system disclosed in Japanese Unexamined Patent Application Publication No. 2018-84911 that it is impossible to propose a work plan by which a worker can achieve both the performance of a task assigned to him/her and an exercise for maintaining his/her good health.

The present disclosure has been made in view of the above-described circumstance, and an object thereof is to provide a schedule management system, its control method, and a control program capable of proposing a work plan by which a user can achieve both the performance of a task assigned to him/her and an exercise for maintaining his/her good health.

A schedule management system according to an aspect of the present disclosure includes: an acquisition unit configured to acquire information about a plurality of task items and information about a target exercise amount of a first user for each predetermined period; a task dividing unit configured to extract at least one task item from among the plurality of task items by referring to a database, and assign the extracted at least one task item to the first user as a task item for the predetermined period, the at least one task item representing a task with which the first user achieves an exercise amount corresponding to his/her target exercise amount for each predetermined period, and in the database, information about, for each of the plurality of task items, an exercise amount per unit time corresponding to that task item being registered; and an output unit that outputs information about the task item assigned to the first user by the task dividing unit. The above-described schedule management system can assign, to a user, a task item with which the user can achieve an appropriate exercise amount. That is, the above-described schedule management system can propose a work plan by which a user can achieve both the performance of a task assigned to him/her and an exercise for maintaining his/her good health.

The acquisition unit may further acquire information about a predetermined task item that should be performed by the first user in the predetermined period, and the task dividing unit may extract at least one task item including the predetermined task item from among the plurality of task items by referring to the database, and assign the extracted at least one task item to the first user as the task item for the predetermined period, the at least one task item representing a task with which the first user achieves the exercise amount corresponding to his/her target exercise amount for each predetermined period.

The acquisition unit may further acquire information about a task item that the first user has determined he/she cannot carry out, and the task dividing unit may extract at least one task item other than the task item that the first user has determined he/she cannot carry out from among the plurality of task items by referring to the database, and assign the extracted at least one task item to the first user as the task item for the predetermined period, the at least one task item representing a task with which the first user achieves the exercise amount corresponding to his/her target exercise amount for each predetermined period.

The acquisition unit may acquire, as feedback information, information about an actual exercise amount performed by the first user as he/she carries out the task item assigned to him/her, and the task dividing unit may extract at least one task item from among the plurality of task items by further referring to the feedback information, and assign the extracted at least one task item to the first user as the task item for the predetermined period, the at least one task item representing a task with which the first user achieves the exercise amount corresponding to his/her target exercise amount for each predetermined period.

The information about the actual exercise amount performed by the first user may be at least one of a heart rate, a pulse rate, the number of steps, a calorie consumption of the first user acquired by a wearable terminal attached to the first user, and an amount of change in a weight of the first user measured by a scale.

The target exercise amount may be a target calorie consumption of the first user.

The target exercise amount may be determined based on a difference between a target weight and a current weight of the first user.

The target exercise amount may be determined based on a difference between a target body fat percentage and a current body fat percentage of the first user.

The target exercise amount may be determined based on a difference between a target body shape and a current body shape of the first user.

The output unit may display information about the task item assigned to the first user on a monitor.

The plurality of task items may be those assigned to a predetermined group to which a plurality of users including the first user belong; the acquisition unit may acquire, for each of the plurality of users belonging to the predetermined group, information about his/her target exercise amount for each predetermined period; the task dividing unit may extract, for each of the plurality of users, at least one task item from among the plurality of task items by referring to the database, and assign the extracted at least one task item to that user as the task item for the predetermined period, the at least one task item representing a task with which that user achieves the exercise amount corresponding to his/her target exercise amount for each predetermined period; and the output unit may output information about the task items assigned to the users by the task dividing unit.

The task dividing unit may extract, for each of the plurality of users, at least one task item from among the plurality of task items by referring to the database, and assign the extracted at least one task item to that user as the task item for the predetermined period, the at least one task item representing a task with which that user achieves an exercise amount corresponding to a ratio of his/her target exercise amount to a sum total of target exercise amounts of all users.

The plurality of task items may be those that should be performed in the predetermined period by the predetermined group, and the task dividing unit may divide all of the plurality of task items among the plurality of users.

A method for controlling a schedule management system according to an aspect of the present disclosure includes: acquiring information about a plurality of task items and information about a target exercise amount of a first user for each predetermined period; extracting at least one task item from among the plurality of task items by referring to a database, the at least one task item representing a task with which the first user achieves an exercise amount corresponding to his/her target exercise amount for each predetermined period, and in the database, information about, for each of the plurality of task items, an exercise amount per unit time corresponding to that task item being stored; assigning the extracted at least one task item to the first user as a task item for the predetermined period; and outputting information about the task item assigned to the first user. By the above-described method for controlling a schedule management system, it is possible to assign, to a user, a task item with which the user can achieve an appropriate exercise amount. That is, by the above-described method for controlling a schedule management system, it is possible to propose a work plan by which a user can achieve both the performance of a task assigned to him/her and an exercise for maintaining his/her good health.

A control program according to an aspect of the present disclosure causes a computer to perform; a process for acquiring information about a plurality of task items and information about a target exercise amount of a first user for each predetermined period; a process for extracting at least one task item from among the plurality of task items by referring to a database, the at least one task item representing a task with which the first user achieves an exercise amount corresponding to his/her target exercise amount for each predetermined period, and in the database, information about, for each of the plurality of task items, an exercise amount per unit time corresponding to that task item being stored; a process for assigning the extracted at least one task item to the first user as a task item for the predetermined period; and a process for outputting information about the task item assigned to the first user. By the above-described control program, it is possible to assign, to a user, a task item with which the user can achieve an appropriate exercise amount. That is, by the above-described control program, it is possible to propose a work plan by which a user can achieve both the performance of a task assigned to him/her and an exercise for maintaining his/her good health.

According to the present disclosure, it is possible to provide a schedule management system, its control method, and a control program capable of proposing a work plan by which a user can achieve both the performance of a task assigned to him/her and an exercise for maintaining his/her good health.

The above and other objects, features and advantages of the present disclosure will become more fully understood from the detailed description given hereinbelow and the accompanying drawings which are given by way of illustration only, and thus are not to be considered as limiting the present disclosure.

### BRIEF DESCRIPTION OF DRAWINGS

FIG. 1 is a block diagram showing an example of a configuration of a schedule management system according to a first embodiment:

FIG. 2 is a block diagram showing an example of a configuration of a schedule management apparatus provided in the schedule management system shown in FIG. 1;

FIG. 3 is a flowchart showing operations performed by the schedule management system shown in FIG. 1;

FIG. 4 is a conceptual diagram showing an example of an exercise amount per unit time for each of task items registered in a database;

FIG. 5 is a conceptual diagram for explaining an example of operations performed by the schedule management system shown in FIG. 1:

FIG. 6 is a conceptual diagram for explaining another example of operations performed by the schedule management system shown in FIG. 1:

FIG. 7 is a block diagram showing a modified example of the schedule management system shown in FIG. 1;

FIG. 8 is a conceptual diagram for explaining an example of operations performed by the schedule management system shown in FIG. 7; and

FIG. 9 is a conceptual diagram for explaining another example of operations performed by the schedule management system shown in FIG. 7.

### DESCRIPTION OF EMBODIMENTS

The present disclosure will be described hereinafter through embodiments of the disclosure, but the invention according to the claims is not limited to the below-shown embodiments. Further, all the components/structures described in the embodiments are not necessarily indispensable as means for solving the problem. For clarifying the explanation, the following description and the drawings are partially omitted and simplified as appropriate. The same reference numerals (or symbols) are assigned to the same elements throughout the drawings and redundant descriptions thereof are omitted as appropriate.

### First Embodiment

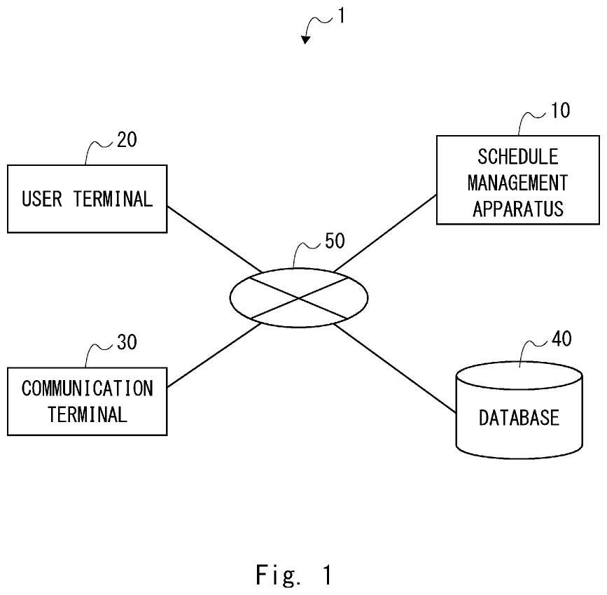

FIG. 1 is a block diagram showing an example of a configuration of a schedule management system 1 according to a first embodiment. A schedule management system 1 extracts at least one task item, which represents a task with which a user achieves an amount of exercise (hereinafter also referred to as an “exercise amount”) corresponding to his/her target exercise amount for each predetermined period, from among a plurality of task items by referring to a database, and assigns the extracted at least one task item to the user as a task item for the predetermined period (for each predetermined period, i.e., for a respective predetermined period). In this way, the schedule management system 1 can assign, to the user, a task item representing a task with which he/she can achieve an appropriate exercise amount. That is, the schedule management system 1 can propose a work plan by which a user can achieve both the performance of a task assigned to him/her and an exercise for maintaining his/her good health. Details of the schedule management system 1 and the like will be described hereinafter.

As shown in FIG. 1, the schedule management system 1 includes a schedule management apparatus 10, a user terminal 20, a communication terminal 30, a database 40, and a network 50. The schedule management apparatus 10 can also be referred to as a schedule management system. The schedule management apparatus 10, the user terminal 20, the communication terminal 30, and the database 40 are configured so that they can communicate with each other through a wired or wireless network 50.

The user terminal 20 is a terminal capable of performing communication, owned by or temporarily assigned to a user U1, such as a PC (Personal Computer) terminal, a mobile terminal such as a smartphone or a tablet-type terminal, or a dedicated communication terminal prepared for the schedule management system. For example, the user U1 enters information about a target exercise amount of the user U1 for each predetermined period to the user terminal 20 by operating an operation screen, a keyboard. or the like of the user terminal 20. The user terminal 20 receives the information about the target exercise amount of the user U1 for each predetermined period, entered by the operation performed by the user U1, and transmits the received information to the schedule management apparatus 10 through the network 50.

Note that the information about the target exercise amount of the user U1 for each predetermined period is information about an exercise amount that the user U1 wants to achieve for each predetermined period. Note that the predetermined period can be arbitrarily set and is, for example, one week, one month, or three months. For example, the target exercise amount of the user U1 for each predetermined period is expressed by a target calorie consumption of the user U1 for each predetermined period, but is not limited to this example. That is, it is expressed by a target amount of decrease in weight (a difference between a current body weight and a target body weight), a target amount of decrease in body fat percentage (a difference between a current body fat percentage and a target body fat percentage), or a target degree of change in body shape (a degree of change in the appearance of his/her body or the like). Alternatively, the user terminal 20, the schedule management apparatus 10, or the like may derive (e.g., calculate) a target calorie consumption from a target amount of decrease in weight, a target amount of decrease in body fat percentage, or a target degree of change in body shape, or the like. Note that it is preferred to further refer to information about at least one of the age, gender, height, and weight of the user U1 when the target calorie consumption is derived. In this way, it is possible to derive the target calorie consumption more reliably.

Note that the user terminal 20 may receive not only the information about the target exercise amount of the user U1 for each predetermined period. but also information about a predetermined task item that should be performed in the predetermined period by the user U1 and/or information about a task item that the user U1 has determined he/she cannot carry out, and transmit these received information items to the schedule management apparatus 10 through the network 50. Further, the user terminal 20 may also receive information about at least one of the above-described age, gender, height, and weight of the user U1, and transmit the received information to the schedule management apparatus 10 through the network 50.

Note that the information about the predetermined task item that should be carried out by the user U1 for each predetermined period is, for example, a meeting the user U1 himself/herself has to participate in or checking of emails transmitted to the user U1. Further, the information about the task item that the user U1 determines cannot carry out is a task that the user U1 is not qualified to perform (e.g., a delivery task by a user U1 who does not possess a driver's license) or a task that is difficult for the user U1 to perform due to an injury or the like.

Further, although an example in which the user terminal 20 receives the information about the target exercise amount of the user U1 for each predetermined period and transmits the received information to the schedule management apparatus 10 through the network 50 is described in this embodiment, the present disclosure is not limited to this example. For example, a communication terminal other than the user terminal 20, such as a communication terminal 30 (which will be described later), may receive the information about the target exercise amount of the user U1 for each predetermined period and transmit the received information to the schedule management apparatus 10 through the network 50.

The communication terminal 30 is a terminal capable of performing communication, owned by or temporarily assigned to a manager X1, such as a PC terminal, a mobile terminal such as a smartphone or a tablet-type terminal, or a dedicated communication terminal prepared for the schedule management system. For example, the manager X1 enters information about a plurality of task items assigned to a predetermined group G1 to which the user U1 belongs to the communication terminal 30 by operating an operation screen, a keyboard, or the like of the communication terminal 30. The communication terminal 30 receives the information about the plurality of task items assigned to the predetermined group G1, entered by the operation performed by the manager X1, and transmits the received information to the schedule management apparatus 10 through the network 50.

Note that the manager X1 is, for example, a leader of the predetermined group G1 to which the user U1 belongs or a person who assigns tasks to the members of the predetermined group G1. The predetermined group G1 is, for example, a department or the like of a company or the like to which the user U1 belongs.

Note that although an example case where a plurality of task items assigned to the predetermined group G1 are task items that should be performed by the predetermined group G1 in each predetermined period is described in this embodiment, the present disclosure is not limited to this example.

Further, although an example case where the communication terminal 30 receives information about a plurality of task items assigned to the predetermined group G1 and transmits the received information to the schedule management apparatus 10 through the network 50 is described in this embodiment, the present disclosure is not limited to this example. For example, a communication terminal other than the communication terminal 30, such as the user terminal 20, may receive information about a plurality of task items assigned to the predetermined group G1 and transmits the received information to the schedule management apparatus 10 through the network 50. Further, the plurality of task items transmitted to the schedule management apparatus 10 are not limited to those that are assigned to the predetermined group G1.

The schedule management apparatus 10 extracts at least one task item, which represents a task with which the user U1 achieves an exercise amount corresponding to his/her target exercise amount for each predetermined period, from among the plurality of task items assigned to the group G1, to which the user U1 belongs, by referring to the database 40, in which information about, for each of the plurality of task items, an exercise amount per unit time (such as a calorie consumption per unit time) corresponding to that task item is registered, and assigns the extracted at least one task item to the user U1 as a task item for the predetermined period (for each predetermined period, i.e., for a respective predetermined period).

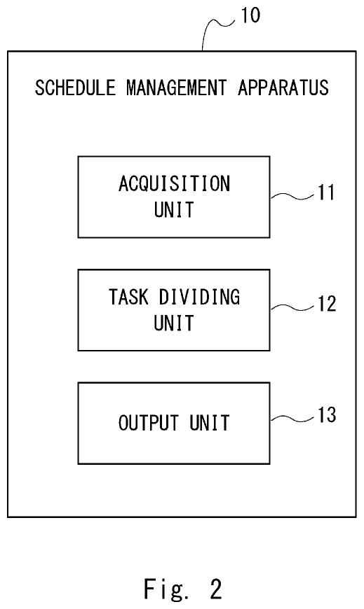

FIG. 2 is a block diagram showing an example of a configuration of the schedule management apparatus 10. As shown in FIG. 2, the schedule management apparatus 10 includes at least an acquisition unit 11, a task dividing unit 12, and an output unit 13.

Firstly, the acquisition unit 11 acquires information about a plurality of task items assigned to the predetermined group G1, transmitted from the communication terminal 30 or the like. The acquisition unit 11 further acquires information about a target exercise amount of the user U1 for each predetermined period, transmitted from the user terminal 20 or the like.

The task dividing unit 12 extracts at least one task item, which represents a task with which the user U1 achieves an exercise amount corresponding to the target exercise amount for each predetermined period, from among the plurality of task items assigned to the predetermined group G1 by referring to the database 40, and assigns the extracted at least one task item to the user U1 as a task item for the predetermined period.

The output unit 13 outputs information about the task item assigned to the user U1. The information output from the output unit 13 is, for example, transferred to the user terminal 20 through the network 50 and displayed on a monitor of the user terminal 20. In this way, the user U1 can know the task item assigned to himself/herself.

Note that when the schedule management apparatus 10 acquires information about a predetermined task item that should be performed in each predetermined period by the user U1, it extracts at least one task item which includes this predetermined task and represents a task with which the user U1 achieves an exercise amount corresponding to his/her target exercise amount for each predetermined period, and assigns the extracted at least one task item to the user U1 as the task item for the predetermined period. Further, when the schedule management apparatus 10 acquires information about a predetermined task item that the user U1 has determined he/she cannot carry out, it extracts at least one task which is different from the task item that the user has determined he/she cannot carry out and represents a task with which the user U1 achieves an exercise amount corresponding to his/her target exercise amount for each predetermined period, and assigns the extracted at least one task item to the user U1 as the task item for the predetermined period.

As described above, the schedule management system 1 according to this embodiment extracts at least one task item, which represents a task with which the user U1 achieves an exercise amount corresponding to his/her target exercise amount for each predetermined period, from among the plurality of task items assigned to the predetermined group G1 by referring to the database 40, and assigns the extracted at least one task item to the user U1 as a task item for the predetermined period. In this way, the schedule management system 1 according to this embodiment can assign, to the user U1, a task item which represents a task with which the user U1 can achieve an appropriate exercise amount. That is, the schedule management system 1 according to this embodiment can propose a work plan by which the user U1 achieves both the performance of a task assigned to him/her and an exercise for maintaining his/her good health.

Note that in the schedule management apparatus 10, the acquisition unit 11 may further acquire, as feedback information, information about an actual exercise amount performed by the user U1 as he/she carries out the task item assigned to him/her. For example, the acquisition unit 11 acquires, as feedback information, information about a heart rate, a pulse rate, the number of steps, or a calorie consumption of the user U1 measured by a wearable terminal (not shown) attached to the user U1, and/or acquires, as feedback information, information about an amount of change in the weight of the user U1 measured by a scale (not shown). In this case, the task dividing unit 12 extracts at least one task item, which represents a task with which the user U1 achieves an exercise amount corresponding to his/her target exercise amount for each predetermined period by referring not only to the database 40 but also to the feedback information. In this way, the task dividing unit 12 can extract at least one task item, which represents a task with which the user U1 achieves an exercise amount corresponding to his/her target exercise amount for each predetermined period, more accurately. Note that the information registered in the database 40 may be updated by the feedback information.

### (Operation of Schedule Management System 1)

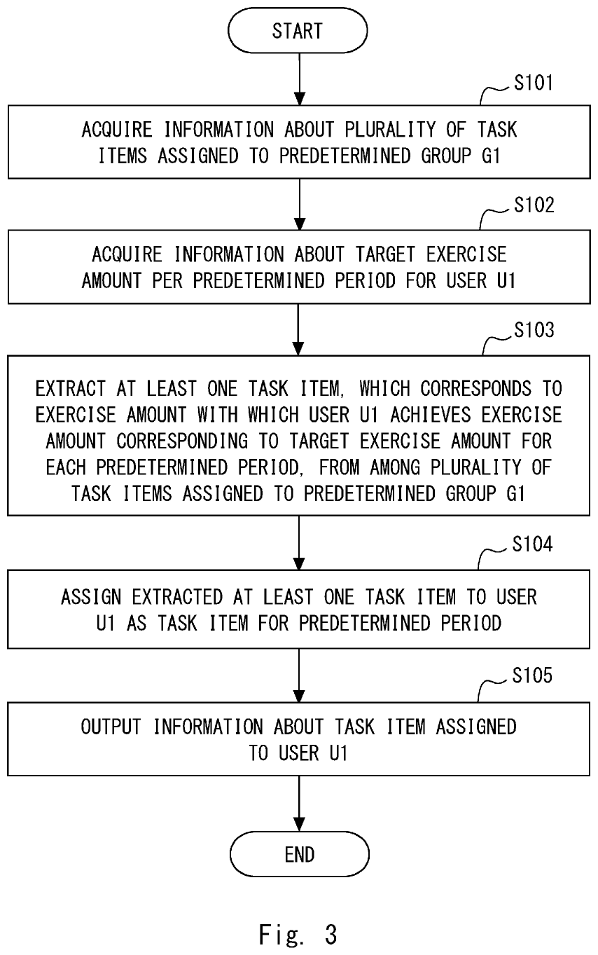

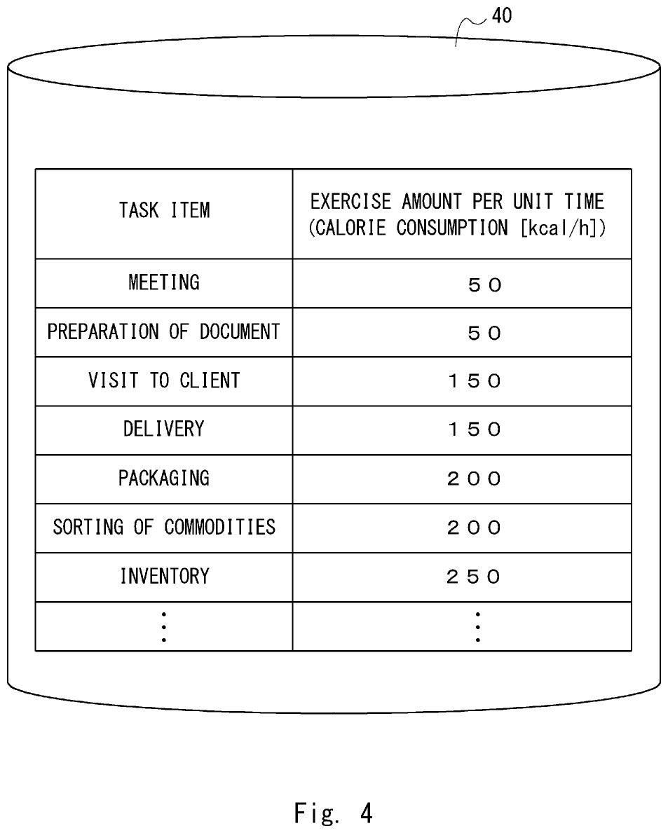

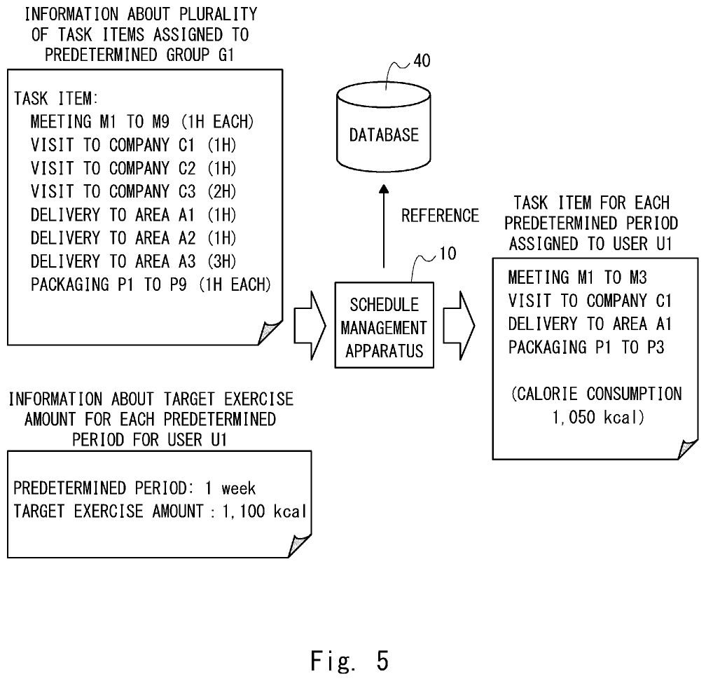

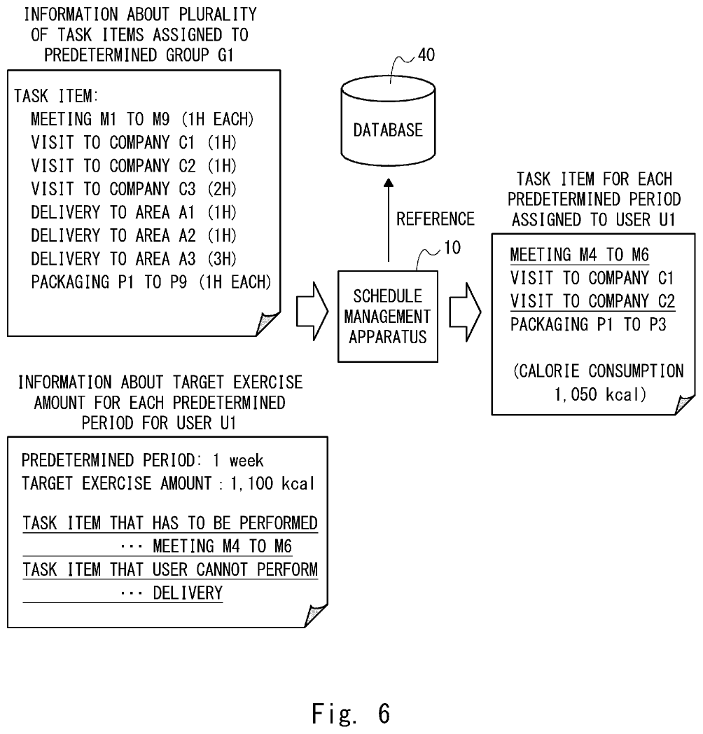

Next, operations performed by the schedule management system 1 will be described with reference to FIGS. 3 to 6. FIG. 3 is a flowchart showing operations performed by the schedule management system 1. FIG. 4 is a conceptual diagram showing an example of an exercise amount per unit time for each of task items registered in the database 40. FIG. 5 is a conceptual diagram for explaining an example of operations performed by the schedule management system 1. FIG. 6 is a conceptual diagram for explaining another example of operations performed by the schedule management system 1. Note that in the examples shown in FIGS. 4 to 6, calorie consumptions are used as examples of the exercise amount.

As show in FIG. 4, in the database 40, the following information and the like are registered; a calorie consumption per hour for a meeting is 50 kcal; a calorie consumption per hour for preparation of documents is 50 kcal; a calorie consumption per hour for a visit to a client is 150 kcal; a calorie consumption per hour for a delivery is 150 kcal; a calorie consumption per hour for packaging is 200 kcal; a calorie consumption per hour for sorting of commodities is 200 kcal; and a calorie consumption per hour for inventory is 250 kcal.

Firstly, the communication terminal 30 receives information about a plurality of task items assigned to a predetermined group G1, entered by an operation performed by a manager X1, and transmits the received information to the schedule management apparatus 10 through the network 50.

Specifically, in the example shown in FIG. 5, the plurality of tasks assigned to the predetermined group G1 include meetings M1 to M9 (one hour each), a visit to a company C1 (one hour), a visit to a company C2 (one hour), a visit to a company C3 (two hours), a delivery to an area A1 (one hour), a delivery to an area A2 (one hour), a delivery to an area A3 (three hours), and packaging P1 to P9 (one hour each).

Further, the user terminal 20 receives information about a target exercise amount of a user U1 for each predetermined period, entered by an operation performed by the user U1, and transmits the received information to the schedule management apparatus 10 through the network 50.

In the example shown in FIG. 5, the predetermined period is set to one week, and a target calorie consumption, which is the target exercise amount of the user U1 for each week, is set to 1,100 kcal.

In the schedule management apparatus 10, the acquisition unit 11 first acquires information about the plurality of task items assigned to the predetermined group G1, transmitted from the communication terminal 30 or the like (Step S101). The acquisition unit 11 further acquires information about the target exercise amount of the user U1 for each predetermined period, transmitted from the user terminal 20 or the like (Step S102).

After that, in the schedule management apparatus 10, the task dividing unit 12 extracts at least one task item, which represents a task with which the user U1 achieves an exercise amount corresponding to his/her target exercise amount for each predetermined period, from among the plurality of task items assigned to the predetermined group G1 by referring to the database 40 (Step S103). Then, the task dividing unit 12 assigns at least one extracted task item to the user U1 as a task item for the predetermined period (Step S104).

In the example shown in FIG. 5, the schedule management apparatus 10 extracts task items including the participation in the meetings M1 to M3, the visit to the company C1, the delivery to the area A1. and the packaging P1 to P3 so that the calorie consumption of the user U1 for each week becomes equal to or close to his/her target calorie consumption, and assigns the extracted task items to the user U1 as the task items for the predetermined period. Note that as shown in the database 40 shown in FIG. 4. the total calorie consumption of the task items assigned to the user U1 is 1,050 kcal, and is close to the target calorie consumption of the user U1, which is 1,100 kcal.

After that, the output unit 13 outputs information about the task items assigned to the user U1 (Step S105). The information output from the output unit 13 is, for example, transferred to the user terminal 20 through the network 50 and displayed on a monitor of the user terminal 20. In this way, the user U1 can know the task items assigned to himself/herself.

Note that as already described above, the user terminal 20 may receive not only the information about the target exercise amount of the user U1 for each predetermined period, but also information about a predetermined task item that should be performed in the predetermined period by the user U1 and/or information about a task item that the user U1 has determined he/she cannot carry out, and transmit these received information items to the schedule management apparatus 10 through the network 50. This feature will be briefly described hereinafter with reference to FIG. 6.

In the example shown in FIG. 6, the target calorie consumption, which is the target exercise amount of the user U1 for each week, is set to 1,100 kcal. Further, the participation in the meetings M4 to M6 is set as a mandatory task item for the user U1. Further, the delivery is set as a task item that the user U1 cannot carry out. In this case, in consideration of the set conditions, the schedule management apparatus 10 extracts the task items including the participation in the meetings M4 to M6, the visit to the company C1. the visit to the company C2, and the packaging P1 to P3 so that the calorie consumption of the user U1 for each week becomes equal to or close to his/her target calorie consumption, and assigns the extracted task items to the user U1 as the task items for each week. That is, the schedule management apparatus 10 assigns, to the user U1, the participation in the meetings M4 to M6 instead of the participation in the meetings M1 to M3, and the visit to the company C2 instead of the delivery to the area A1. At this point, as shown in the database 40 shown in FIG. 4, the total calorie consumption of the task items assigned to the user U1 is 1,050 kcal, and is close to the target calorie consumption of the user U1, which is 1100 kcal.

As describe above, the schedule management system 1 according to this embodiment extracts at least one task item, which represents a task with which the user U1 achieves an exercise amount corresponding to his/her target exercise amount for each predetermined period, from among the plurality of task items assigned to the predetermined group G1 by referring to the database 40, and assigns the extracted at least one task item to the user U1 as a task item for the predetermined period. In this way, the schedule management system 1 according to this embodiment can assign, to the user U1, a task item which represents a task with which the user U1 can achieve an appropriate exercise amount. That is, the schedule management system 1 according to this embodiment can propose a work plan by which the user U1 achieves both the performance of a task assigned to him/her and an exercise for maintaining his/her good health.

### (Modified Example of Schedule Management System 1)

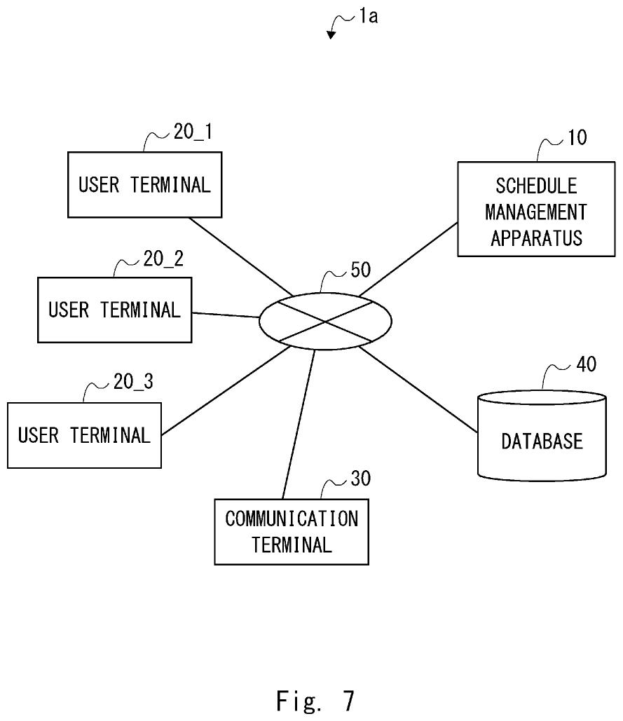

Note that although an example case where the schedule management apparatus 10 extracts at least one task item, which represents a task with which the user U1 achieves an exercise amount corresponding to his/her target exercise amount for each predetermined period, from among a plurality of task items assigned to the predetermined group G1, and assigns the extracted at least one task item to the user U1 as a task item for the predetermined period has been described in this embodiment, the present disclosure is not limited to this example. The schedule management apparatus 10 may also extract at least one task item representing a task with which a user, who belongs to the predetermined group G1 but is different from the user U1, achieves an exercise amount corresponding to his/her target exercise amount for each predetermined period, and assign the extracted at least one task item to that user as a task item for the predetermined period. This feature will be briefly described hereinafter with reference to FIGS. 7 to 9.

FIG. 7 is a block diagram showing a modified example of the schedule management system 1 as a schedule management system 1a. The schedule management system 1a includes a schedule management apparatus 10, three user terminals 20, a communication terminal 30, a database 40, and a network 50. Hereafter, the three user terminals 20 are referred to as user terminals 20_1, 20_2 and 20_3, respectively. Note that although three user terminals 20 are provided in the example shown in FIG. 7, the number of user terminals is not limited to three. That is, the number of user terminals 20 provided in the schedule management system is any number larger than one.

The user terminals 20_1 to 20_3 are terminals capable of performing communication, owned by or temporarily assigned to users U1 to U3, respectively. The user terminal 20_1 receives information about a target exercise amount of the user U1 for each predetermined period, entered by an operation performed by the user U1, and transmits the received information to the schedule management apparatus 10 through the network 50. The user terminal 20_2 receives information about a target exercise amount of the user U2 for each predetermined period, entered by an operation performed by the user U2, and transmits the received information to the schedule management apparatus 10 through the network 50. The user terminal 20_3 receives information about a target exercise amount of the user U3 for each predetermined period, entered by an operation performed by the user U3, and transmits the received information to the schedule management apparatus 10 through the network 50.

The schedule management apparatus 10 extracts, for each of the users U1 to U3, at least one task item, which represents a task with which that user achieves an exercise amount corresponding to his/her target exercise amount for each predetermined period, from among the plurality of task items assigned to the predetermined group G1 by referring to the database 40, and assigns, to each of the users U1 to U3, the extracted at least one task item to that user as a task item for the predetermined period.

More specifically, in the schedule management apparatus 10, the acquisition unit 11 first acquires information about a plurality of task items assigned to a predetermined group G1, transmitted from the communication terminal 30 or the like. The acquisition unit 11 further acquires information about, for each of the users U1 to U3, his/her target exercise amount for each predetermined period, transmitted from a respective one of the user terminals 20_1 to 20_3.

After that, in the schedule management apparatus 10, the task dividing unit 12 extracts, for each of the users U1 to U3. at least one task item, which represents a task with which that user achieves an exercise amount corresponding to his/her target exercise amount for each predetermined period, from among the plurality of task items assigned to the predetermined group G1 by referring to the database 40, and assigns, for each of the users U1 to U3, the extracted at least one task item to that user as a task item for the predetermined period.

Note that the task dividing unit 12 may assign all of the plurality of tasks assigned to the predetermined group G1 to the users U1 to U3 while dividing these tasks among the users U1 to U3. Further, when the sum total of exercise amounts of the plurality of task items assigned to the predetermined group G1 is smaller than the sum total of the target exercise amounts of the users U1 to U3, the task dividing unit 12 may extract, for each of the users U1 to U3, at least one task item representing a task with which that user achieves an exercise amount corresponding to a ratio of his/her target exercise amount to the sum total of the target exercise amounts of all users U1 to U3.

The output unit 13 outputs information about the task items assigned to the users U1 to U3, respectively. The information output from the output unit 13 is, for example, transferred to the user terminals 20_1 to 20_3 through the network 50 and displayed on monitors thereof. In this way, each of the users U1 to U3 can know the task item assigned to himself/herself.

### (Example of Operation of Schedule Management System 1a)

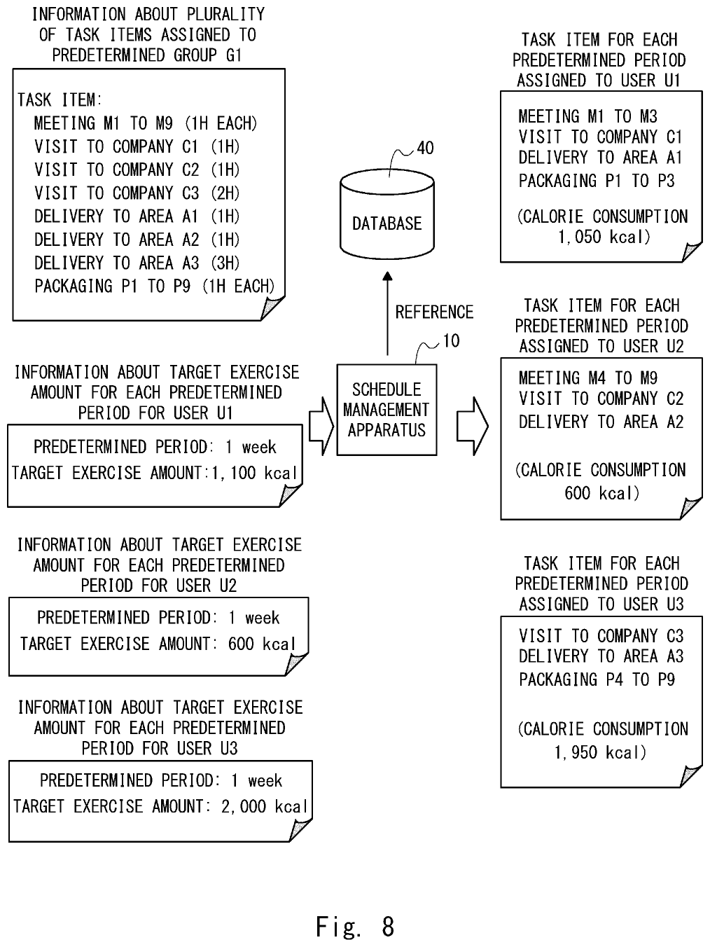

FIG. 8 is a conceptual diagram for explaining an example of operations performed by the schedule management system 1a. Specifically, in the example shown in FIG. 8, the plurality of tasks assigned to the predetermined group G1 include meetings M1 to M9 (one hour each), a visit to a company C1 (one hour), a visit to a company C2 (one hour), a visit to a company C3 (two hours), a delivery to an area A1 (one hour), a delivery to an area A2 (one hour), a delivery to an area A3 (three hours), and packaging P1 to P9 (one hour each).

Further, in the example shown in FIG. 8, the predetermined period is set to one week for all users. Further, the target calorie consumption of the user U1, which is his/her target exercise amount for each week, is set to 1,100 kcal. The target calorie consumption of the user U2, which is his/her target exercise amount for each week, is set to 600 kcal. The target calorie consumption of the user U3, which is his/her target exercise amount for each week, is set to 2,000 kcal.

Note that the schedule management apparatus 10 extracts task items including the participation in the meetings M1 to M3, the visit to the company C1, the delivery to the area A1, and the packaging P1 to P3 so that the calorie consumption of the user U1 for each week becomes equal to or close to his/her target calorie consumption, and assigns the extracted task items to the user U1 as the task items for the predetermined period. Note that as shown in the database 40 shown in FIG. 4, the total calorie consumption of the task items assigned to the user U1 is 1,050 kcal, and is close to the target calorie consumption of the user U1, which is 1,100 kcal.

Further, the schedule management apparatus 10 extracts task items including the participation in the meetings M4 to M9, the visit to the company C2, and the delivery to the area A2 so that the calorie consumption of the user U2 for each week becomes equal to or close to his/her target calorie consumption, and assigns the extracted task items to the user U2 as the task items for the predetermined period. Note that as shown in the database 40 shown in FIG. 4, the total calorie consumption of the task items assigned to the user U2 is 600 kcal, and is equal to the target calorie consumption of the user U2, which is 600 kcal.

Further, the schedule management apparatus 10 extracts task items including the visit to the company C3, the delivery to the area A3, and the packaging P4 to P9 so that the calorie consumption of the user U3 for each week becomes equal to or close to his/her target calorie consumption, and assigns the extracted task items to the user U3 as the task items for the predetermined period. Note that as shown in the database 40 shown in FIG. 4, the total calorie consumption of the task items assigned to the user U3 is 1,950 kcal, and is close to the target calorie consumption of the user U3, which is 2,000 kcal.

### (Other Example of Operation of Schedule Management System 1a)

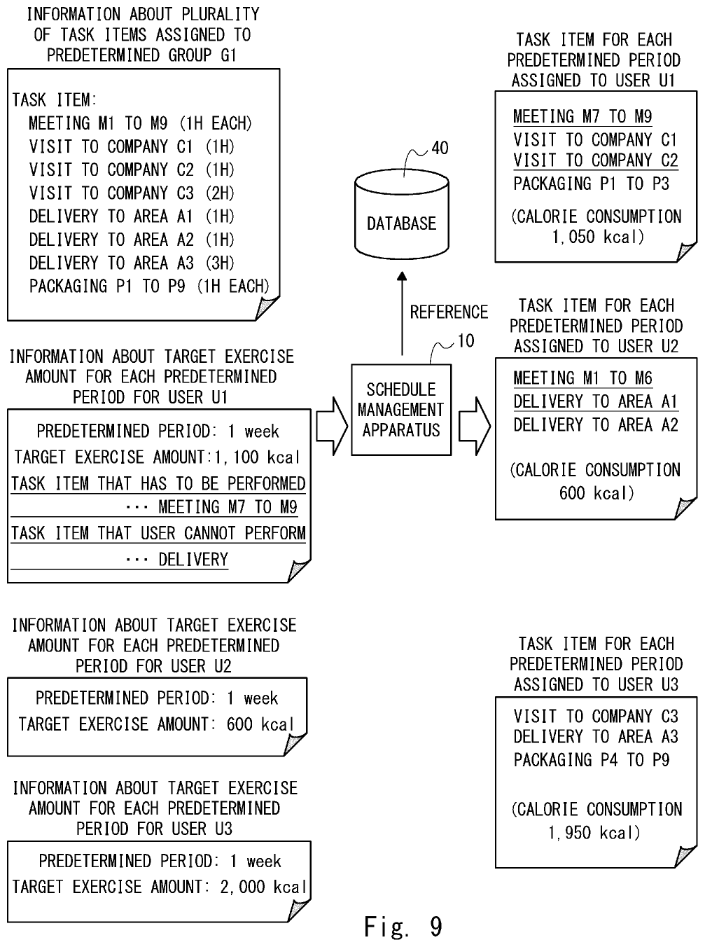

FIG. 9 is a conceptual diagram for explaining another example of operations performed by the schedule management system 1a. In the example shown in FIG. 9, the target calorie consumption, which is the target exercise amount of the user U1 for each week, is set to 1,100 kcal. Further, the participation in the meetings M7 to M9 is set as a mandatory task item for the user U1, and the delivery is set as a task item that the user U1 cannot carry out.

In this case, in consideration of the set conditions, the schedule management apparatus 10 extracts the task items including the participation in the meetings M7 to M9, the visit to the company C1. the visit to the company C2, and the packaging P1 to P3 so that the calorie consumption of the user U1 for each week becomes equal to or close to his/her target calorie consumption, and assigns the extracted task items to the user U1 as the task items for each week. That is, the schedule management apparatus 10 assigns, to the user U1, the participation in the meetings M7 to M9 instead of the participation in the meetings M1 to M3, and the visit to the company C2 instead of the delivery to the area A1. Note that as shown in the database 40 shown in FIG. 4. the total calorie consumption of the task items assigned to the user U1 is 1,050 kcal, and is close to the target calorie consumption of the user U1, which is 1,100 kcal.

Further, the schedule management apparatus 10 extracts task items including the participation in the meetings M1 to M6, the delivery to the area A1, and the delivery to the area A2 so that the calorie consumption of the user U2 for each week becomes equal to or close to his/her target calorie consumption, and assigns the extracted task items to the user U2 as the task items for each week. That is, the schedule management apparatus 10 assigns, to the user U2, the participation in the meetings M1 to M3 instead of the participation in the meetings M7 to M9, and the delivery to the area A1 instead of the visit to the company C2. Note that as shown in the database 40 shown in FIG. 4, the total calorie consumption of the task items assigned to the user U2 is 600 kcal, and is equal to the target calorie consumption of the user U2, which is 600 kcal.

The task items assigned to the user U3 are similar to those in the example shown in FIG. 8, and therefore descriptions thereof are omitted.

As described above, the schedule management system 1a according to this embodiment extracts, for each of the users U1 to U3, at least one task item, which represents a task with which that user achieves an exercise amount corresponding to his/her target exercise amount for each predetermined period, from among the plurality of task items assigned to the predetermined group G1 by referring to the database 40, and assigns, for each of the users U1 to U3, the extracted at least one task item to that user as a task item for the predetermined period. In this way. the schedule management system 1a according to this embodiment can assign, to each of the users U1 to U3, a task item which represents a task with which that user can achieve an appropriate exercise amount. That is, the schedule management system 1a according to this embodiment can propose, to each of the users U1 to U3, a work plan by which that user achieves both the performance of a task assigned to him/her and an exercise for maintaining his/her good health.

Note that the present disclosure is not limited to the above-described embodiments, and they can be modified as appropriate without departing from the scope and spirit of the disclosure.

Further, in the present disclosure, some or all of the control processes performed in the schedule management system can be implemented by having a CPU (Central Processing Unit) execute a computer program.

In the aforementioned program includes instructions (or software codes) that, when loaded into a computer, cause the computer to perform one or more of the functions described in the embodiments. The program may be stored in a non-transitory computer readable medium or a tangible storage medium. By way of example, and not a limitation, the non-transitory computer readable media or tangible storage media can include a random-access memory (RAM), a read-only memory (ROM), a flash memory, a solid-state drive (SSD) or other types of memory technologies, a CD-ROM, a digital versatile disc (DVD), a Blu-ray (Registered Trademark) disc or other types of optical disc storage, and magnetic cassettes, magnetic tape, magnetic disk storage or other types of magnetic storage devices. The program may be transmitted on a transitory computer readable medium or a communication medium. By way of example, and not a limitation, the transitory computer readable media or communication media can include electrical, optical, acoustical, or other forms of propagation signals.

From the disclosure thus described, it will be obvious that the embodiments of the disclosure may be varied in many ways. Such variations are not to be regarded as a departure from the spirit and scope of the disclosure, and all such modifications as would be obvious to one skilled in the art are intended for inclusion within the scope of the following claims.

What is claimed is:

1. A schedule management system comprising:
an acquisition unit configured to acquire information about a plurality of task items and information about a target exercise amount of a first user for each predetermined period;
a task dividing unit configured to extract at least one task item from among the plurality of task items by referring to a database, and assign the extracted at least one task item to the first user as a task item for the predetermined period, the at least one task item representing a task with which the first user achieves an exercise amount corresponding to his/her target exercise amount for each predetermined period, and in the database, information about, for each of the plurality of task items, an exercise amount per unit time corresponding to that task item being registered; and
an output unit that outputs information about the task item assigned to the first user by the task dividing unit.

2. The schedule management system according to claim 1, wherein
the acquisition unit further acquires information about a predetermined task item that should be performed by the first user in the predetermined period, and
the task dividing unit extracts at least one task item including the predetermined task item from among the plurality of task items by referring to the database, and assigns the extracted at least one task item to the first user as the task item for the predetermined period, the at least one task item representing a task with which the first user achieves the exercise amount corresponding to his/her target exercise amount for each predetermined period.

3. The schedule management system according to claim 1, wherein
the acquisition unit further acquires information about a task item that the user has determined he/she cannot carry out, and
the task dividing unit extracts at least one task item other than the task item that the first user has determined he/she cannot carry out from among the plurality of task items by referring to the database, and assigns the extracted at least one task item to the first user as the task item for the predetermined period, the at least one task item representing a task with which the first user achieves the exercise amount corresponding to his/her target exercise amount for each predetermined period.

4. The schedule management system according to claim 1, wherein
the acquisition unit acquires, as feedback information, information about an actual exercise amount performed by the first user as he/she carries out the task item assigned to him/her, and
the task dividing unit extracts at least one task item from among the plurality of task items by further referring to the feedback information, and assigns the extracted at least one task item to the first user as the task item for the predetermined period, the at least one task item representing a task with which the first user achieves the exercise amount corresponding to his/her target exercise amount for each predetermined period.

5. The schedule management system according to claim 4, wherein the information about the actual exercise amount performed by the first user is at least one of a heart rate, a pulse rate, the number of steps, a calorie consumption of the first user acquired by a wearable terminal attached to the first user, and an amount of change in a weight of the first user measured by a scale.

6. The schedule management system according to claim 1, wherein the target exercise amount is a target calorie consumption of the first user.

7. The schedule management system according to claim 1, wherein the target exercise amount is determined based on a difference between a target weight and a current weight of the first user.

8. The schedule management system according to claim 1, wherein the target exercise amount is determined based on a difference between a target body fat percentage and a current body fat percentage of the first user.

9. The schedule management system according to claim 1, wherein the target exercise amount is determined based on a difference between a target body shape and a current body shape of the first user.

10. The schedule management system according to claim 1, wherein the output unit displays information about the task item assigned to the first user on a monitor.

11. The schedule management system according to claim 1, wherein
the plurality of task items are those assigned to a predetermined group to which a plurality of users including the first user belong,
the acquisition unit acquires, for each of the plurality of users belonging to the predetermined group, information about his/her target exercise amount for each predetermined period,
the task dividing unit extracts, for each of the plurality of users, at least one task item from among the plurality of task items by referring to the database, and assigns the extracted at least one task item to that user as the task item for the predetermined period, the at least one task item representing a task with which that user achieves the exercise amount corresponding to his/her target exercise amount for each predetermined period, and
the output unit outputs information about the task items assigned to the users by the task dividing unit.

12. The schedule management system according to claim 11, wherein the task dividing unit extracts, for each of the plurality of users, at least one task item from among the plurality of task items by referring to the database, and assigns the extracted at least one task item to that user as the task item for the predetermined period, the at least one task item representing a task with which that user achieves an exercise amount corresponding to a ratio of his/her target exercise amount to a sum total of target exercise amounts of all users.

13. The schedule management system according to claim 11, wherein
the plurality of task items are those that should be performed in the predetermined period by the predetermined group, and
the task dividing unit divides all of the plurality of task items among the plurality of users.

14. A method for controlling a schedule management system, comprising:
acquiring information about a plurality of task items and information about a target exercise amount of a first user for each predetermined period;
extracting at least one task item from among the plurality of task items by referring to a database, the at least one task item representing a task with which the first user achieves an exercise amount corresponding to his/her target exercise amount for each predetermined period, and in the database, information about, for each of the plurality of task items, an exercise amount per unit time corresponding to that task item being stored;
assigning the extracted at least one task item to the first user as a task item for the predetermined period; and
outputting information about the task item assigned to the first user.

15. A non-transitory computer readable storage medium storing a control program for causing a computer to perform:
a process for acquiring information about a plurality of task items and information about a target exercise amount of a first user for each predetermined period;
a process for extracting at least one task item from among the plurality of task items by referring to a database, the at least one task item representing a task with which the first user achieves an exercise amount corresponding to his/her target exercise amount for each predetermined period, and in the database, information about, for each of the plurality of task items, an exercise amount per unit time corresponding to that task item being stored;
a process for assigning the extracted at least one task item to the first user as a task item for the predetermined period; and
a process for outputting information about the task item assigned to the first user.
